(19) United States
(12) Patent Application Publication
(10) Pub. No.: US 2010/0087516 A1
(43) Pub. Date: Apr. 8, 2010

(54) ANTIBACTERIAL MACROLACTIN A THAT BACILLUS POLYFERMENTICUS KJS-2 PRODUCED IN

(76) Inventors: Jae-Seon Kang, Busan (KR); Chun-Gyu Kim, Gimhae (KR); Dong-Hee Kim, Gimhae (KR); Kang-Min Kim, Busan (KR); Dong-Hun Kim, Busan (KR); Jin-Young Lee, Busan (KR); Guang-Jin Choi, Busan (KR); In-June Cha, Busan (KR); Jae-Sun Hong, Sungnam (KR); Yong-Geun Hong, Changwon (KR)

Correspondence Address: BIRCH STEWART KOLASCH & BIRCH, PO BOX 747, FALLS CHURCH, VA, 22040-0747, US

(21) Appl. No.: 12/531,434

(22) PCT Filed: Mar. 12, 2008
(86) PCT No.: PCT/KR2008/001393

§ 371 Date: Sep. 15, 2009

(30) Foreign Application Priority Data
Mar. 16, 2007 (KR) 10-2007-0026285

(51) Int. Cl. A61K 31/335; A61P 31/12; C12N 1/20

(52) U.S. Cl. 514/450; 435/252.5

(57) ABSTRACT

The present invention relates to uses of Macrolactin A produced by Bacillus polyfermenticus KJS-2 (KCCM 10769P), which is a new bacillus strain, as an antibiotic. Macrolactin A of the present invention, which is produced by Bacillus polyfermenticus KJS-2, shows a broad spectrum of antibiotic activity against a variety of microorganisms and fungi, and is proved to be very efficient for the inhibition of particularly vancomycin-resistant enterococci (VRE) and methicillin-resistant Staphylococcus Aureus (MRSA) that are multidrug-resistant bacteria. The antibiotic Macrolactin A produced by Bacillus polyfermenticus KJS-2, can be used as an excellent antibiotic against vancomycin-resistant enterococci (VRE) and methicillin-resistant Staphylococcus Aureus (MRSA), and thus the present invention is a very useful invention for medical industry.

### TECHNICAL FIELD

The present invention relates to Macrolactin A, which is an antibiotic produced by Bacillus polyfermenticus KJS-2 (KCCM10769P), and its use; specifically to the Macrolactin A having antibiotic activity against harmful bacteria such as vancomycin-resistant enterococci (VRE) and methicillin-resistant Staphylococcus Aureu (MRSA), and its use.

### BACKGROUND ART

The increase of vancomycin-resistant enterococci (VRE) is unfortunate to mankind and has a lot of problems, such as the extremely high cost for the development of a new antibiotic. There was a report that in 1989 where only 0.3% of contagion by VRE were reported in a hospital, but the rate increased to 7.9% in 1993. The fatality of bacteremia caused by multidrug-resistant VRE is as high as around 70%, while there is a worry that the ability of the resistance gene of VRE to transform other gram-positive cocci, which may increase the possibility of vancomycin-resistant MRSA. Recently the antibiotic teycoplanin has begun to be used domestically against resistant bacteria, while the appearance of teycoplanin-resistant bacteria has already been reported.

Therefore, the present inventors have isolated a new bacterial strain, Bacillus polyfermenticus KJS-2, which produces Macrolactin A having antibiotic activity against vancomycin-resistant VRE and methicillin-resistant MRSA as well as against Escherichia coli, Bacillis subtilis 168, Micrococcus luteus, Vibrio vulnificus and Streptocuccus parauberis, and the new strain has been registered (Strain Registration Number KCCM10769P). The active component was purified and its structure was determined to verify that it is Macrolactin A, and proven to have the same effects.

Hereinafter, a summary of the previous studies in respects to Macrolactin A and the strains which produce Macrolactin A will be given.

Macrolactin A was first purified in 1989, by William Fenical, from a marine bacterium existing in the deep sea. It has been reported that Macrolactin A has selective antibacterial activity and shows cytotoxicity on the B16-F10 murine melanoma cancer cell, and that it also has antiviral activity against Herpes simplex and HIV.

In 1997, Macrolactin A was purified from Actinomadura sp. by ICK-DONG YOO, and the purified Macrolactin A was used to study the protection of neurons triggered by glutamate.

In 2001, Macrolactin A was purified from Bacillus. sp. PP19-H3 by Hiroshi Sano, and its antibiotic activity was studied against Staphylococcus aureus IFO 127:2 and Bacillus subtilis IFO 3134.

In 2003, Macrolactin A was purified from Streptomyces sp. YB-401 by Sung-Won Choi, and was shown to have an inhibitory effect on the biosynthesis of cholesterol.

In 2004, Macrolactin A was purified from Bacillus amyloliquefaciens CHO104 by Keun-Hyung Park, and its antibiotic activity was studied against Staphylococcus aureus KCTC 1928, Escherichia coli KCTC 2593 and Botiytis cinerea. In 2005, Macrolactin A was purified from Bacillus sp. sunhua by Joo-Won Suh, and the purified Macrolactin A was used to study the inhibitory effect on Streptomyces scabies.

In 2006, Macrolactin A and Malonyl-macrolactin A (MMA) were purified from Bacillus subtilis DSM 16696 by Gabriella Molinari, each of which was tested for the antibiotic activity against vancomycin-resistant enterococci (VRE), methicillin-resistant Staphylococcus aureu (MRSA) and Burkholderia cepacia. In this study Malonyl-macrolactin A (MMA) was shown to have excellent antibiotic activity against all the bacteria used for the experiment, while Macrolactin A was shown to have antibiotic activity only against MRSA. These results are very meaningful, yet the maximum amount of purified Macrolactin A produced by each strain is less than 1 mg/l and that of malonyl-macrolactin A (MMA) is less than 12 mg/l, which has hindered their industrial application. Further, there has been no study on the optical isomers of Macrolactin A, and low yield thereof makes them difficult to be identified. Studies in this aspect would be necessary in the future, and the Macrolactin obtained as the result of the present invention has also not been studied sufficiently as an optical isomer. Theoretically, the 4 chiral centers in the structure makes possible the existence of 16 optical isomers. As are the cases with most medicines, Macrolactins with optically different structures would show characteristically different effects, even if their structural formulas are the same. This means that the substances produced by different strains may have different effects depending on their optical structures. It is a scientifically proven fact that even the substances with the same structural formula have different properties depending on their optical structures.

### DISCLOSURE OF INVENTION

### Technical Problem

Accordingly, it is an object of the present invention to produce Macrolactin A having antibiotic activity against VRE and MRSA as well as against Escherichia coli (E. coli), Bacillus subtilis 168, Micrococcus luteus, Vibrio vulnificus and Streptococcus parauberis, and to develop the present substance into an antibiotic by testing the effects thereof.

The present substance is an antibiotic of the macrolide class with a molecular weight of 40224 having a ring structure with many double bonds and hydroxyl groups (—OH). The 24-membered ring structure has carbons and oxygens, and the molecular formula of the present substance is C₂₄H₃₄O₅. The present invention is disclosed for the purpose of providing a means of specifically controlling VRE and MRSA, taking the advantage of the strain of Bacillus polyfermenticus KJS-2 (Accession Number KCCM10769P), which is a newly isolated strain.

### Technical Solution

To accomplish one of the objects, the present invention provides a Macrolactin A having excellent antibiotic activity against VRE and MRSA, taking advantage by advantageously using the strain Bacillus polyfermenticus KJS-2 (Accession Number KCCM10769P).

To accomplish another object, the present invention provides a Macrolactin A, which is produced by Bacillus polyfermenticus KJS-2, having excellent antibiotic activity against Escherichia coli (E. coli), Bacillus subtilis 168, Micrococcus luteus, Vibrio vulnificus, and Streptococcus parauberis; as well as Macrolactin derivatives produced by the strain.

### ADVANTAGEOUS EFFECTS

The Macrolactin A produced by Bacillus polyfermenticus KJS-2, which is the new strain provided by the present invention, shows a broad spectrum of antibiotic activity against a variety of microorganisms and fungi.

Remarkably, the average Minimal Inhibitory Concentration required for the inhibition of more than 90% (MIC>90) of the growth of the 11 VRE strains and the 13 MRSA strains is about 31.25 μg/ml and about 19.83 μg/ml, respectively, which is 4 to 5.3 times more activity than that of the teycoplanin currently used for the patients infected with multidrug-resistant bacteria; and thus shows that it is valuable enough to develop into an antibiotic.

Therefore, Macrolactin A produced by Bacillus polyfermenticus KJS-2 and also the derivatives of the Macrolactin A of the present invention, can produce excellent substances for controlling microorganisms and bacteria, which results in being very useful for the medical industry.

### BRIEF DESCRIPTION OF THE DRAWINGS

FIG. 1 is the number of cells in the culture medium of Bacillus polyfermenticus KJS-2, the strain used in the present invention, measured by a UV detector (OD₆₀₀ₙₘ) at various time points during the fermentation.

FIG. 2 is the chromatogram of the culture medium of Bacillus polyfermenticus KJS-2, the strain used in the present invention, taken at various time points during the fermentation and analyzed by HPLC.

FIG. 3 is the LC/Mass analysis data for the culture medium of Bacillus polyfermenticus KJS-2, the strain used in the present invention, which is taken after 2.5 days of fermentation. The medium is extracted and is analyzed by LC/Mass.

FIG. 4 is the bioassay of the culture medium of Bacillus polyfermenticus KJS-2, the strain used in the present invention, which is taken after 25 days of fermentation. The medium is extracted and fractionated by HPLC, and the fractions of each peak were subjected to bioassay.

FIG. 5 is the LC/Mass data to determine the purity and the molecular weight of the substance purified from the 1st fraction that shows excellent antibiotic activity in FIG. 4.

FIG. 6 is the preparative LC analysis of the 1st fraction, in FIG. 4, of the culture medium of Bacillus polyfermenticus KJS-2, which is taken from the substance generated by the fermentation.

FIG. 7 is the LC/Mass data to determine the purity and the molecular weight of the substance purified from the 1st fraction of the preparative LC of FIG. 6.

FIG. 8 is the ¹H-NMR spectrum of the purified substance of FIG. 7, analyzed by the Brucker NMR at 500 MHz.

FIG. 9 is the ¹³C-NMR spectrum of the purified substance of FIG. 7, analyzed by the Brucker NMR at 500 MHz.

FIG. 10 is the DEPT-90 NMR spectrum of the purified substance of FIG. 7, analyzed by the Brucker NMR at 500 MHz.

FIG. 11 is the DEPT-135 NMR spectrum of the purified substance of FIG. 7, analyzed by the Brucker NMR at 500 MHz.

FIG. 12 is the HOMO-COZY NMR spectrum of the purified substance of FIG. 7, analyzed by the Brucker NMR at 500 MHz.

FIG. 13 is the HMQC NMR spectrum of the purified substance of FIG. 7, analyzed by the Brucker NMR at 500 MHz.

FIG. 14 is the HMBC NMR spectrum of the purified substance of FIG. 7, analyzed by the Brucker NMR at 500 MHz.

FIG. 15 is the HR-Mass analysis of the purified substance of FIG. 7.

FIG. 16 is the structural formula of Macrolactin A.

FIG. 17 is the analysis data for the culture medium of Bacillus polyfermenticus KJS-2, the strain used in the present invention, which is taken after 2.5 days of fermentation. The medium is extracted, and the extracted solution is analyzed by LC/Mass.

FIG. 18 is the analysis data for the culture medium of Bacillus polyfermenticus KJS-2, the strain used in the present invention, which is taken after 42 days of fermentation. The medium is extracted, and the extracted solution is analyzed by LC/Mass.

FIG. 19 is the HPLC analysis to measure indirectly the solubility of Macrolactin A in the 50% acetone solvent and the methanol solvent.

(A) is the HPLC chromatogram of 1 μl aliquot of 1 mg of Macrolactin A in 1 ml of 50% acetone that has been eluted 10 times.

(B) is the HPLC chromatogram of 1 μg aliquot of 1 mg of Macrolactin A in 1 ml of methanol that has been diluted 10 times.

FIG. 20 is the result of antibiotic activity of purified Macrolactin A tested against Saccharomyces cerevisiae, Vibrio vulnificus, Micrococcus luteus and Streptococcus parauberis.

FIG. 21 is the result of antibiotic activity of purified Macrolactin A tested at a gradient of concentration against the strain of VRE5.

### BEST MODE FOR CARRYING OUT THE INVENTION

A strain that has different morphology from that of the other strains of bacillus came to be isolated in the course of experiments for antibiotic activity against the Bacillus polyfermenticus n. sp, which had been isolated by Dr. Terakado's group in Japan in 1933. Microscopic observation revealed that the strain has the characteristics of bacillus and forms a spore, and the analysis of genealogical diagram based on the homology in the DNA sequence of 16s rRNA proved that it is a new strain belonging to the genus Bacillus.

The bacillus strain is named Bacillus polyfermenticus KJS-2, which was deposited in the KCCM (Korea Culture Center of Microorganisms) on Aug. 16, 2006 and given an Accession Number KCCM10769P.

To purify the substance that has antibiotic activity produced by the strain of Bacillus polyfermenticus KJS-2, the strain of Bacillus polyfermenticus KJS-2 was cultured in 3 L of TSB medium (TSB agar: Tryptone: 17 g, Soytone: 3 g, Dextrose: 25 g, NaCl: 5 g, Dipotassium Phosphate: 25 g, pH 6.8 to 7.2), which was then inoculated into the same medium and fermented for 25 days (30° C., 200 rpm, 1 vvm, pH6.8). The culture medium was subjected to solvent extraction by ethyl acetate, followed by LC/MS analysis. The fractions with excellent antibiotic activity were searched out by LC/Mass analysis and also tested for antibiotic effects, and eventually subjected to purification using a preparative silicagel RP-18.

Also the first NMR and the second NMR (¹H-NMR, ¹³C-NMR, 90-DEPT, 135-DEPT, HMQC, HMBC) as well as HRMS/FAB were performed to analyze the structure of the finally purified substance, and the result confirmed that the antibiotic substance produced by the strain of Bacillus polyfermenticus KJS-2 of the present invention was Macrolactin A.

The Minimal Inhibitory Concentration of the Macrolactin A, produced by the Bacillus polyfermenticus KJS-2, required for the inhibition of more than 90% (MIC_{>90}) of the growth of the 11 VRE strains and the 13 MRSA strains, which were clinically isolated, was experimentally determined to be about 31.25 μg/ml and about 19.83 μg/ml, respectively, which has 4 to 5.3 times higher activity than that of the teycoplanin currently used to treat multidrug-resistant bacteria. In addition, Macrolactin A showed an excellent antibiotic activity against Escherichia coli, Bacillus subtilis, Micrococcus luteus 168, Vibrio vulnificus and Streptococcus parauberis. The Microlactin A produced by the strain of Bacillus polyfermenticus KJS-2 also showed excellent heat stability and was very stable in weak acidic and neutral environments.

Besides the antibiotic Macrolactin A produced by Bacillus polyfermenticus KJS-2, the derivatives of the substance also showed a broad spectrum of antibiotic activity.

The comprisal of the present invention is described in more detail hereafter with the Experimental Example 1 and Examples below, but the scope of the claim of the present invention is not limited to the Examples below.

### Example 1

### Isolation and Identification of Bacillus polyfermenticus KJS-2 and Production of Antibiotic Substance

[Step 1: Isolation and Identification of Bacillus polyfermenticus KJS-2]

A strain that has different morphology from that of the other strains of bacillus came to be isolated in the course of experiments for antibiotic activity against the Bacillus polyfermenticus n. sp, which had been isolated by Dr. Terakado's group in Japan in 1933. Microscopic observation revealed that the strain has the characteristics of bacillus and forms a spore, and the analysis of genealogical diagram based on the homology in the DNA sequence of 16s rRNA proved that it is a new strain belonging to the genus Bacillus. The present inventors proved that the base sequence of the 16s rRNA of the present strain had a 99% homology with that of the strain of Bacillus sp. PP19-H3, which produced previously known Macrolactin A (Korean Patent Application No. 10-2006096935:2006.10.02)

TABLE 1
Antibiotic activity of the strain of Bacillus polyfermenticus KJS-2

| Strain | Inhibition action |
|---|---|
| Micrococcus luteus | +++ |
| Bacillus subtilis | ++ |
| Aspergillus oryzae | + |
| Aspergillus niger | + |

+++: very strong inhibition
++: strong inhibition
+: inhibition

The present strain, which showed excellent antibiotic activity by itself in Table 1 above, was named Bacillus polyfermenticus KJS-2 and was deposited in the KCCM (Korea Culture Center of Microorganisms) on Aug. 16, 2006 and given an Accession Number KCCM10769P.

[Step 2: Equipment and Conditions for Analysis]

The equipment and conditions described below were used to analyze the antibiotic substance produced by the strain of the present invention. For HPLC analysis, the agilent 1100 series and Shimadzu HPLC were employed along with the Zorbax SB-C18 column (column size 4.6*250 mm, particle size 5 μm). The solvent inducing 0.1% formic acid added to acetonitrile and water was used.

Two conditions were used for HPLC analysis: (1) a gradient concentration of acetonitrile from 0% to 100% for 20 minutes, (2) an isocratic concentration of acetonitrile at 40%. The flow rate for HPLC was 1 ml/min using Agilent 1100 series, and 15 ml/min using Shimadzu HPLC. A UV detector was used for HPLC analysis at the wavelengths of 228, 262, 280, 300 and 350 nm. The agilent 1100 MSD was employed for LC/Mass analysis, and the conditions for the LC/mass analysis was the same as HPLC, and the conditions for LC/Mass analysis is as follows: in AP-ESI mode, the flow rate of drying gas was 13 l/min, the vapor pressure was 50 psi, the temperature of drying gas was 350° C., the capillary voltage was 4000 V at cation mode and 3500 V at anion mode, the mass range was between 100 and 1000 m/z, fragment voltage was 150 V, and flow rate was 1 ml/min. Agilent preparative LC was used for preparative LC along with a preparative column Gemini-C18 (column size 10 mm*250 mm, particle size 10 μm). Acetonitrile and water were used as solvents with a flow rate of 5 ml/min. A UV detector was used also for the preparative LC at the wavelengths of 228, 262, 280, 300 and 350 nm.

[Step 3: Analysis of the Metabolites of Bacillus polyfermenticus KJS-2]

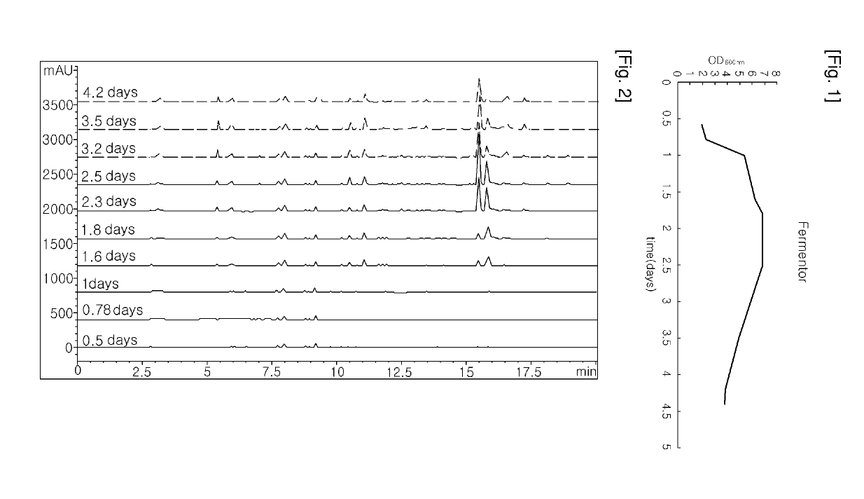

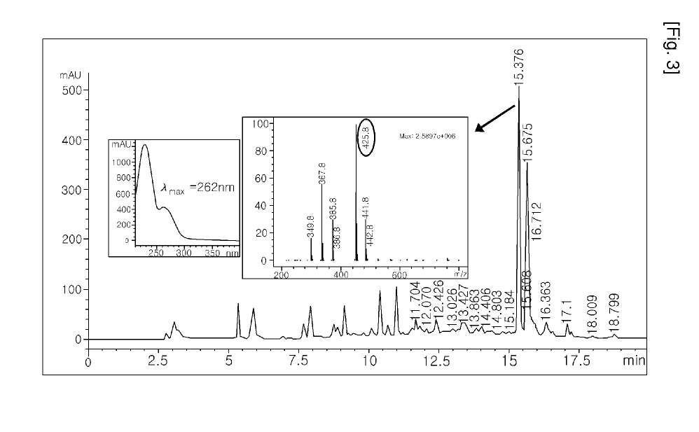

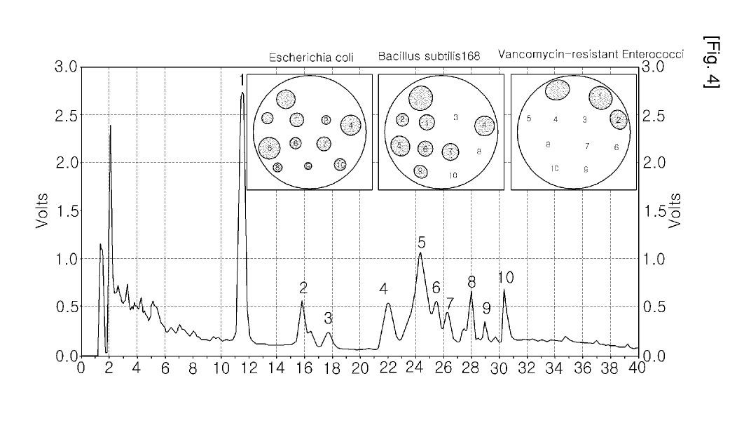

In order to purify the antibiotic substance from the strain of the present invention, the seed culture of Bacillus polyfermenticus KJS-2 was diluted in 3 L of TSB medium (TSB agar; Tryptone 17 g, Soytone 3 g, Dextrose 2.5 g, NaCl 5 g, Dipotassium Phosphate 25 g, pH 6.8 to 72) to have a final concentration of 4% and fermented for 4.5 days (30° C., 200 rpm, 1 vvm, pH6.8). A 3 ml aliquot of the culture medium was taken every 12 hours to measure the amount of fermenting cells using a UV detector (OD₆₀₀ₙₘ, refer to FIG. 1), and a 50 ml aliquot of the culture medium was extracted with ethyl acetate to analyze the metabolites produced by the strain of the present invention, using LC/Mass under the conditions described in Step 3 of EXAMPLE 1. The metabolites produced by the strain of the present invention showed different patterns of chromatogram depending on the length of fermentation time, as shown in FIG. 2. Below, FIG. 3 shows the result of LC/Mass analysis of the culture medium after 25 days of fermentation, where the substance with a retention time of 15.376 min was determined to have a maximum absorption wavelength of 262 nm upon UV analysis, the [M+Na]+ of 425.8 and a molecular weight of 402.8.

### Example 2

### Purification of Antibiotic Substance from the Culture Medium of Bacillus polyfermenticus KJS-2

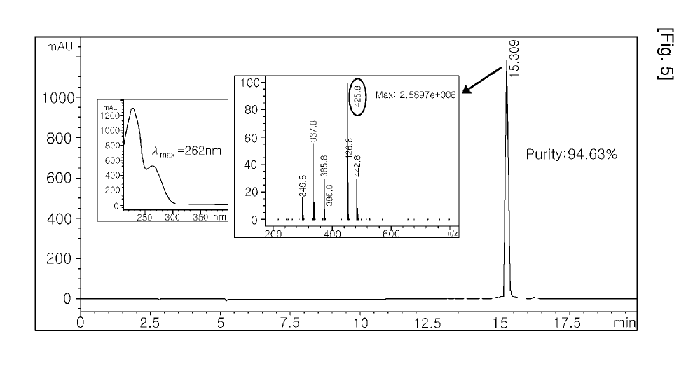

In order to purify the antibiotic substance produced by strain Bacillus polyfermenticus KJS-2, the seed culture of the strain was diluted into 3 L of TSB medium (TSB agar: Tryptone 17 g, Soytone 3 g, Dextrose 2.5 g, NaCl 5 g, Dipotassium Phosphate 2.5 g, pH 6.8 to 72 to a final concentration of 4% and was cultured for 2.5 days (30° C., 200 rpm, 1 vvm, pH6.8). The culture medium was extracted with acetyl acetate and analyzed by HPLC under the conditions using a solvent of Step 2 of Example 1, and each peak was fractionated as in FIG. 4. Each fraction was tested for antibiotic activity against Escherichia coli, Bacillus subtilis 168 and Vancomycin-resistant Enterococci. The result indicated that fractions 1, 4, 5, and 7 had antibiotic activity against Escherichia coli (refer to FIG. 4), and the fractions 1, 2, 4, 5, 6, 7, and 9 against Bacillus subtilis 168 (Refer to FIG. 4). While fractions 1 and 2 both showed antibiotic activity against Vancomycin-resistant Enterococci (refer to FIG. 4). Fraction 1 was determined to be used for experiments, however, because fraction 1 of the present invention not only gave a much higher yield than fraction 2 but it also showed antibiotic activity against all of the three bacteria used for the experiment (refer to FIG. 4). FIG. 5 below is the analysis of fraction 1 of FIG. 4 by LC/Mass under the same conditions of Step 2 of Example 1, and the purification was to a purity of 94.63%.

### Example 3

### Structural Analysis of the Fraction which Showed Excellent Antibiotic Activity Against VRE

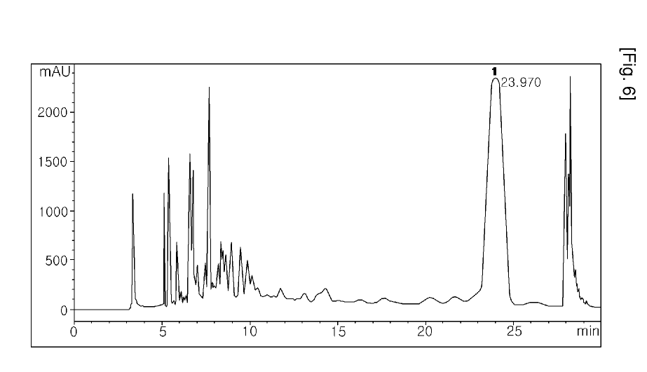

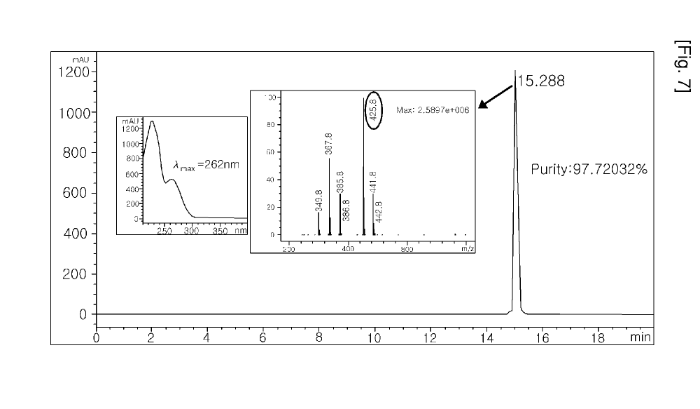

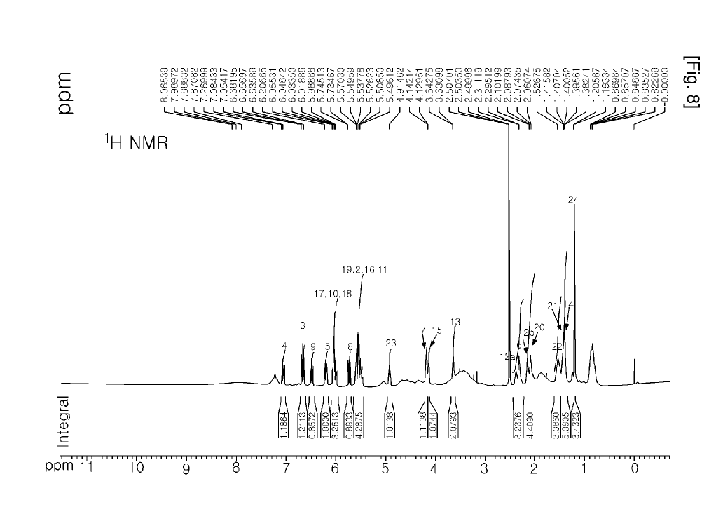

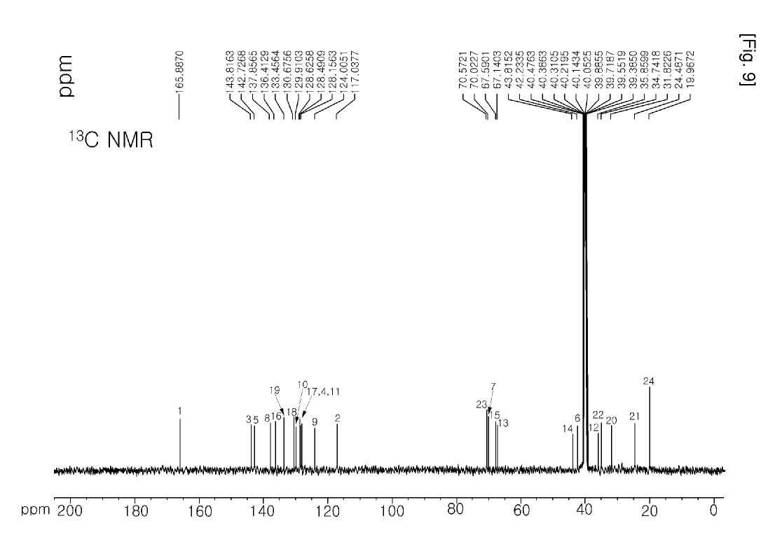

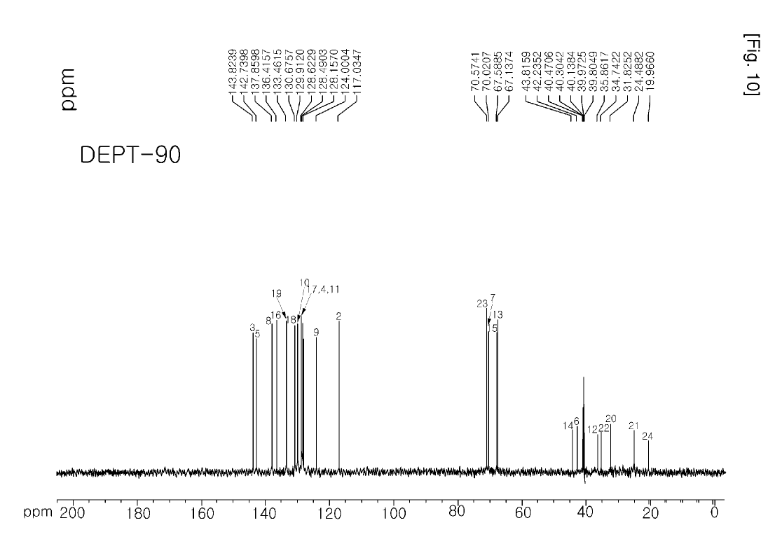

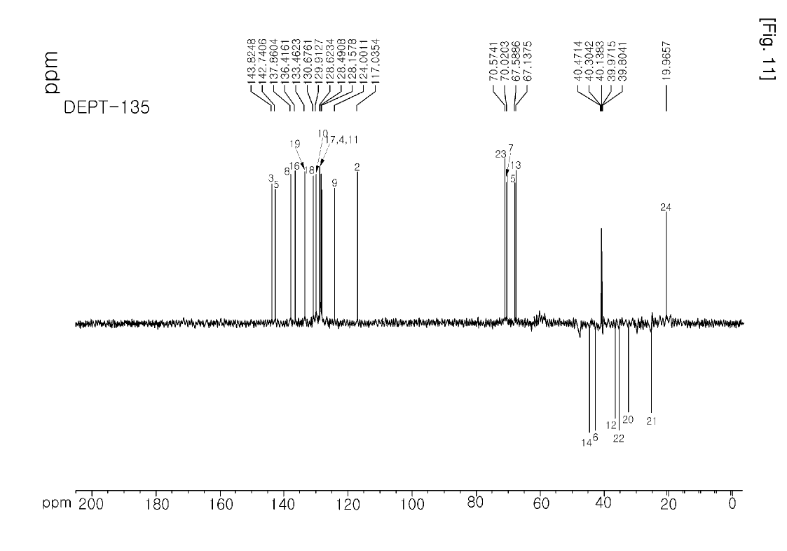

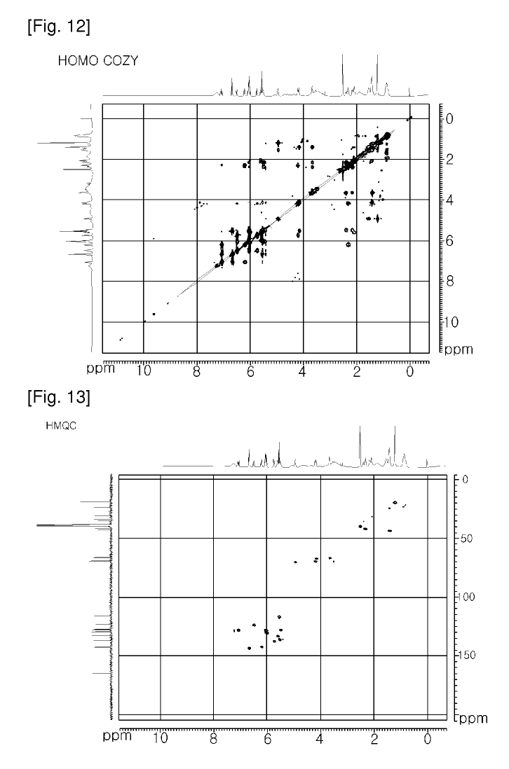

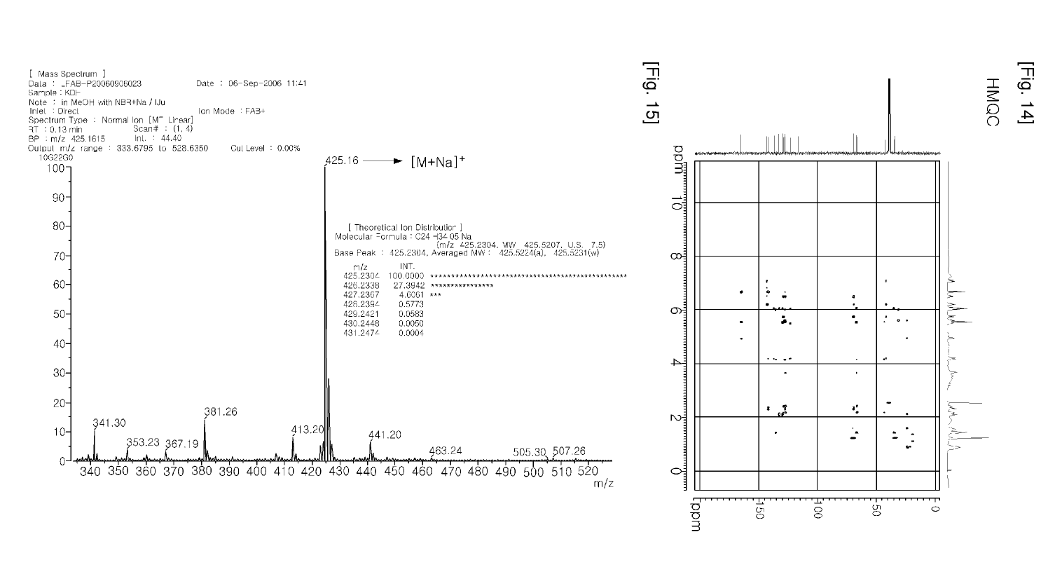

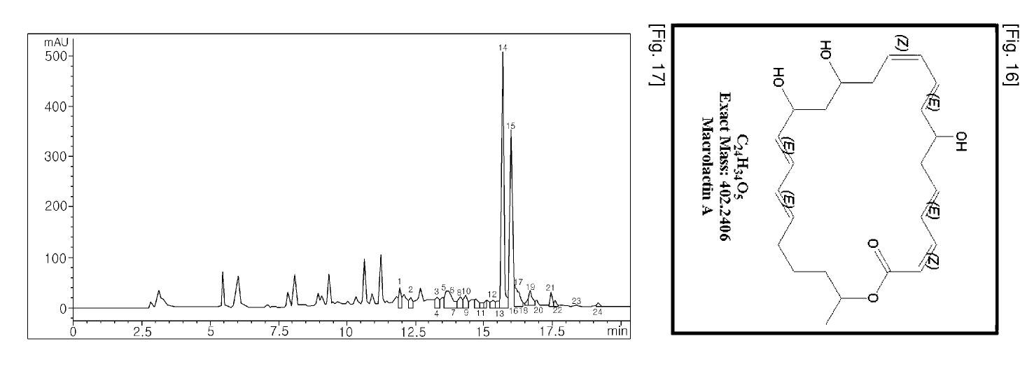

To analyze the structure of the finally purified substance that inhibit the growth of Escherichia coli, Bacillus subtilis 168 and Vancomycin-resistant Enterococci, fractions were prepared in large scale amount under the same conditions for preparative LC of Step 2 of Example 1 (refer to FIG. 6). The fractions were analyzed under the same conditions for LC/Mass of Step 2 of Example 1, and fraction 1 of the Example 2 was subjected to purification having a purity of 97.72% (refer to FIG. 7). 30 mg of the substance purified by preparative LC was dissolved in 700 μl of the solvent DMSO-d6 and subjected to testing of the first and the second NMR (¹H-NMR, ¹³C-NMR, 90-DEPT, 135-DEPT, H-H COZY, HMQC, HMBC). The results of NMR analysis are shown in Table 2, FIG. 8, FIG. 9, FIG. 10, FIG. 11, FIG. 12, FIG. 13 and FIG. 14 below. The finally purified substance was identified to be Macrolactin A based on the results of NMR analysis (refer to FIG. 16), and proven to be a Macrolactin A by performing an HRMS/FAB test, which has the [M+Na]+ of 42523 m/z and the molecular weight of 402.23. HRMS (FAB) JMS-700 was employed for precise mass analysis. The precise mass analysis data by HRMS/FAB is shown in FIG. 15 below. In summary of all the analytical data, fraction 1 of Example 2 having excellent antibiotic activity, was identified as Macrolactin A, which has a molecular formula of C₂₄H₃₄O₅ and a molecular weight of 402.23 (refer to FIG. 16).

TABLE 2
NMR data of Macrolactin A, which is an antibiotic substance
produced by Bacillus polyfermenticus KJS-2

| No. | δH(500 MHz) | m | ∫[Hz] | δH(125 MHz) | | HMBC |
|---|---|---|---|---|---|---|
| 1 | | | | 165.887 | C | |
| 2 | 5.55 | d | 11.476 | 117.038 | CH | C 1, 4 |
| 3 | 6.65 | dd | 11.148, 11.476 | 143.816 | CH | C 1, 4, 5 |
| 4 | 7.06 | dd | 11.148, 15.28 | 128.491 | CH | C 3, 6 |
| 5 | 6.19 | m | | 142.727 | CH | C 3, 4, 6, 7 |
| 6 | 2.32 | m | | 42.2335 | CH2 | C 4, 5, 7, 8 |
| 7 | 4.16 | m | | 70.0227 | CH | C 5, 6, 8, 9 |
| 8 | 5.71 | dd | 5.352, 15.42 | 137.857 | CH | C 6, 7, 10, 11 |
| 9 | 6.48 | dd | 10.134, 15.42 | 124.005 | CH | C 7, 8, 10, 11 |
| 10 | 6.02 | dd | 10.134, 10.9 | 129.91 | CH | C 8, 9, 12 |
| 11 | 5.49 | m | | 128.156 | CH | C 9, 12 |
| 12a | 2.36 | m | | 35.8599 | CH2 | C 10, 11, 13 |
| 12b | 2.14 | m | | | | C 10, 11, 13, 14 |
| 13 | 3.64 | tt | 5.887, 6.293 | 67.1403 | CH | C 11, 15 |
| 14 | 1.41 | m | | 43.8152 | CH2 | C 15, 16 |
| 15 | 4.14 | m | | 67.5901 | CH | C 13, 14, 16, 17 |
| 16 | 5.49 | dd | 6.306, 15.372 | 136.413 | CH | C 14, 15, 17, 18, 19 |
| 17 | 6.04 | dd | 10.353, 15.372 | 128.626 | CH | C 18, 19 |
| 18 | 5.96 | dd | 10.353, 14.88 | 130.676 | CH | C 16, 17 |
| 19 | 5.59 | dt | 14.88, 14.182 | 133.456 | CH | C 17, 20, 21 |
| 20 | 2.07 | m | | 31.8226 | CH2 | C 18, 19, 21, 22 |
| 21 | 1.44 | m | | 24.4871 | CH2 | C 19 |
| 22 | 1.52 | m | | 34.7418 | CH2 | C 21, 23 |
| 23 | 4.9 | m | | 70.5721 | CH | C 1, 21 |
| 24 | 1.2 | d | 7.4 | 19.9672 | CH3 | C 22, 23 |

### Example 4

### LC/Mass Analysis of the Metabolites in the Culture Medium of Bacillus polyfermenticus KJS-2

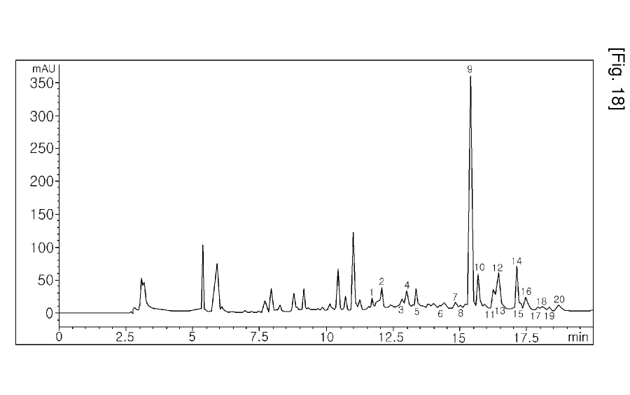

50 ml of the culture medium of Bacillus polyfermenticus KJS-2 of Example 2 was extracted by using ethyl acetate after 2.5 days and 42 days of fermentation and analyzed by LC/Mass. The results shown in Table 3 and Table 4 below (refer to FIG. 17 and FIG. 18) were obtained based on the molecular weights and UV spectrum of all the metabolites produced by Bacillus polyfermenticus KJS-2 as well as the existing documents about the metabolites of the genus Bacillus. The suggestion of the prospected substances in Table 3 and Table 4 below were based on the molecular weights and the characteristic UV wavelengths of Macrolactin A derivatives, and additional data are required in the future regarding the structural analysis by NMR as well as the tests for antibiotic activity.

TABLE 3
LC/Mass analysis of the extracted solution of the culture medium
of Bacillus polyfermenticus KJS-2 after 25 days of fermentation

| # of peaks | Retention time | Area | Height | Molecular weight(m/z) | λmax(nm) | The predicted materials |
|---|---|---|---|---|---|---|
| 1 | 11.704 | 250.5 | 38.3 | | | |
| 2 | 12.07 | 142.8 | 19.3 | 420.8 | 260 | Macrolactinic acid |
| 3 | 12.426 | 142.5 | 25.7 | 564.8 | 268 | Macrolactin B or C |
| 4 | 12.918 | 61.3 | 14.5 | | | |
| 5 | 13.026 | 167.9 | 19.7 | 420.8, 664.8 | 264 | Isomacrolactinic acid & Macrolactin D |
| 6 | 13.427 | 518.2 | 33.2 | 402.8, 564.8 | 266~274 | Macrolactin derivatives, Macrolactin B or C |
| 7 | 13.702 | 71.1 | 12.1 | | | |
| 8 | 13.863 | 202.2 | 21.2 | 402.8 | | Macrolactin derivatives |
| 9 | 14.256 | 144 | 14.7 | 376.8 | 272 | Macrolactin H |
| 10 | 14.408 | 180.1 | 16 | 402.8 | | Macrolactin derivatives |
| 11 | 14.803 | 149.6 | 12.7 | 402.8 | 260 | Macrolactin derivatives |
| 12 | 15.034 | 129.4 | 12.5 | 402.8 | 276 | Macrolactin derivatives |
| 13 | 15.184 | 79.6 | 11.8 | | | |
| 14 | 15.376 | 3104 | 509.6 | 402.8 | 262 | Macrolactin A |
| 15 | 15.675 | 1482 | 338.2 | 488.8 | 260 | Malonyl macrolactin A |
| 16 | 15.712 | 479.7 | 214.3 | 502.8 | | Succinyl macrolactin A |
| 17 | 15.808 | 441.2 | 43.1 | | | |
| 18 | 16.28 | 31.7 | 8.2 | | | |
| 19 | 16.363 | 165 | 26 | 400.8 | 262 | Macrolactin E |
| 20 | 16.584 | 68.8 | 11.8 | | | |
| 21 | 17.119 | 184.3 | 29.2 | 402.8 | 260 | |
| 22 | 17.262 | 74.7 | 12.1 | 402.8, 488.8 | 260 | Macrolactin derivatives, Malonyl macrolactin A |
| 23 | 18.009 | 39.1 | 3.1 | | | |
| 24 | 18.799 | 62.9 | 7.3 | | | |

TABLE 4
LC/Mass analysis of the extracted solution of the culture medium of
Bacillus polyfermenticus KJS-2 after 4.2 days of fermentation

| # of peaks | Retention time | Area | Height | Molecular weight(m/z) | λmax(nm) | The predicted materials |
|---|---|---|---|---|---|---|
| 1 | 11.701 | 59.4 | 12.9 | | | |
| 2 | 12.061 | 94.8 | 22.8 | 420.8 | 260 | Macrolactinic acid |
| 3 | 12.819 | 38.1 | 7.7 | 402.8 | 270 | Macrolactin derivative |
| 4 | 13.008 | 116 | 19.9 | 420.8, 664.8 | 264 | Isomacrolactinic acid & Macrolactin D |
| 5 | 13.34 | 124 | 24.2 | 402.8 | 266 | Macrolactin derivatives |
| 6 | 14.229 | 15.2 | 2.8 | 376.8 | 272 | Macrolactin H |
| 7 | 14.816 | 42.1 | 6.8 | 402.8 | 260 | Macrolactin derivatives |
| 8 | 15.021 | 12.2 | 2.3 | 402.8 | 276 | Macrolactin derivatives |
| 9 | 15.373 | 1957 | 347.6 | 402.8 | 262 | Macrolactin A |
| 10 | 15.671 | 245 | 47.1 | 488.8 | 260 | Malonyl macrolactin A |
| 11 | 16.255 | 171 | 29 | 502.8 | 260 | Succinyl macrolactin A |
| 12 | 16.414 | 87.6 | 25.5 | 400.8 | 262 | Macrolactin E |
| 13 | 16.453 | 185 | 52.4 | 502.8 | 260 | Succinyl macrolactin A |
| 14 | 17.115 | 311 | 58.5 | 402.8 | 260 | Macrolactin derivative |
| 15 | 17.254 | 27.5 | 7 | 402.8, 488.8 | 260 | Macrolactin derivatives, Malonyl macrolactin A |
| 16 | 17.444 | 135 | 16 | 560.8 | 274 | Oxydifficidins |
| 17 | 17.898 | 28 | 4.8 | 502.8, 560.8 | 260 | Succinyl macrolactin A, Oxydifficidins |
| 18 | 18.088 | 43.7 | 5.1 | 502.8, 560.8 | 260 | Succinyl macrolactin A, Oxydifficidins |
| 19 | 18.32 | 22.1 | 4 | 416.8 | 260 | Macrolactin M |
| 20 | 18.676 | 72.6 | 7.4 | 560.8 | 274 | Oxydifficidins |

### Example 5

### Comparison of the Minimal Inhibitory Concentration (MIC) against VRE and MRSA

The MIC (minimal inhibitory concentration) against VRE and MRSA was measured to test antibiotic activity of Macrolactin A produced by Bacillus polyfermenticus KJS-2, which is the strain of the present invention, against the two strains. The strains used for the experiment were 11 VRE strains and 13 MRSA strains, which were clinically isolated. Each strain used for the experiment was cultured in MH II medium at 200 rpm for 6 hours at 37° C., the absorbance of the medium measured at OD₆₀₀ₙₘ using a UV detector, and then 1 ml aliquot of the medium was spread over MH II agar medium and incubated for 16 hours at 37° C. The number of colonies formed on the agar medium after 16 hours of incubation was counted. Each strain used for experiment was cultured in MH II medium at 200 rpm for 6 hours at 37° C., and the absorbance of the medium was measured at OD₆₀₀ₙₘ using a UV detector. The number of cells were calculated based on the ratio of the number of colonies to the absorbance of the medium, and measured by the above process. Each culture medium was diluted to the final cell concentration of 0.25*107 cfu/ml and Macrolactin A was dissolved in the solvent DMSO, while ampicillin, teycoplanin, vancomycin and methillin were dissolved in water. To measure the MIC of Macrolactin A or of the four antibiotics, the mixture of the materials listed in Table 5 and Macrolactin A or the mixture of the materials listed in Table 6 and each of the four antibiotics, respectively in a 500 μl eppendorff tub, was incubated at 200 rpm for 16 hours at 37° C., and then absorbance was measured by a UV/Visible Light detector at 600 nm using a Fluorescence Multi-Detection Reader. The result revealed, as in Table 7 and Table 8, that the average MIC_{>90} of Macrolactin A against the 11 VRE strains was 310 μg/ml, which was 4 times higher than that of teycoplanin; while the average MIC>90 of Macrolactin A against the 13 MRSA strains was 19.83 μg/ml, which was 5.3 times higher than that of teycoplanin.

TABLE 5
Comparison of MIC, and the Macrolactin A concentration gradient

| Number | Stock | Final | DMSO | Antibiotics | Water | Cell | Medium | Total |
|---|---|---|---|---|---|---|---|---|
| of tube | concentration | concentration | (μl) | (μl) | (μl) | (μl) | (μl) | (μl) |
| 1 | | | 1 | | 19 | 0 | 80 | 100 |
| 2 | | | 1 | | 19 | 5 | 75 | 100 |
| 3 | 100 | 1000 | | 1 | 19 | 5 | 75 | 100 |
| 4 | 50 | 500 | | 1 | 19 | 5 | 75 | 100 |
| 5 | 25 | 250 | | 1 | 19 | 5 | 75 | 100 |
| 6 | 12.5 | 125 | | 1 | 19 | 5 | 75 | 100 |
| 7 | 6.25 | 62.5 | | 1 | 19 | 5 | 75 | 100 |
| 8 | 3.125 | 31.25 | | 1 | 19 | 5 | 75 | 100 |
| 9 | 1.5625 | 15.625 | | 1 | 19 | 5 | 75 | 100 |
| 10 | 0.78125 | 7.8125 | | 1 | 19 | 5 | 75 | 100 |
| 11 | 0.390625 | 3.90625 | | 1 | 19 | 5 | 75 | 100 |
| 12 | 0.1953125 | 1.953125 | | 1 | 19 | 5 | 75 | 100 |

Macrolactin A solubilized in DMSO*
The concentration of cell* is 0.25 × 10⁷ cfu/ml

TABLE 6
Comparison of MIC, and the concentration gradients of
ampicillin, teicoplanin, vancomycin and methicillin.

| | Stock | Final | | | | | |
|---|---|---|---|---|---|---|---|
| Number | concentration | concentration | Water | Antibiotics | Cell | Medium | Total |
| of tube | (μg/μl) | (μg/ml) | (μl) | (μl) | (μl) | (μl) | (μl) |
| 1 | | | 20 | | 0 | 80 | 100 |
| 2 | | | 20 | | 5 | 75 | 100 |
| 3 | 5 | 1000 | | 20 | 5 | 75 | 100 |
| 4 | 2.5 | 500 | | 20 | 5 | 75 | 100 |
| 5 | 1.25 | 250 | | 20 | 5 | 75 | 100 |
| 6 | 0.625 | 125 | | 20 | 5 | 75 | 100 |
| 7 | 0.3125 | 62.5 | | 20 | 5 | 75 | 100 |
| 8 | 0.15625 | 31.25 | | 20 | 5 | 75 | 100 |
| 9 | 0.078125 | 15.625 | | 20 | 5 | 75 | 100 |
| 10 | 0.0390625 | 7.8125 | | 20 | 5 | 75 | 100 |
| 11 | 0.01953125 | 3.90625 | | 20 | 5 | 75 | 100 |
| 12 | 0.009765625 | 1.953125 | | 20 | 5 | 75 | 100 |

Ampicillin, teicoplanin, vancomycin methicillin solubilized in water the concentration of cell* is 0.25 × 10⁷ cfu/ml

TABLE 7
Comparison of MIC_{>90} of macrolactin A and other antibiotics against 11 VRE strains

| 11VRE | MIC_{>90}(μg/ml) | | | | |
|---|---|---|---|---|---|
| strains | Macrolactin | Vancomycin | Ampicillin | Teicoplanin | Methicillin |
| VRE1 | 31.25 | 500 | 250 | 250 | — |
| VRE2 | 31.25 | 500 | 250 | 250 | — |
| VRE3 | 31.25 | 125 | 250 | 62.5 | — |
| VRE4 | 31.25 | 250 | 250 | 125 | — |
| VRE5 | 15.63 | 250 | 250 | 125 | — |
| VRE6 | 31.25 | 250 | 250 | 125 | — |
| VRE7 | 31.25 | 250 | 250 | 125 | — |
| VRE9 | 31.25 | 250 | 250 | 62.5 | — |
| VRE10 | 31.25 | 125 | 250 | 62.5 | — |
| VRE914 | 62.5 | 125 | 250 | 62.5 | — |
| VRE915 | 15.63 | 250 | 500 | 125 | — |
| Average | 31.25090909 | 261.3636364 | 272.7272727 | 125 | — |

TABLE 8
Comparison of the MIC_{>90} of Macrolactin A
and other antibiotics against 13 MRSA strains

| 13MRSA | MIC_{>90}(μg/ml) | | | | |
|---|---|---|---|---|---|
| strains | Macrolactin | Vancomycin | Ampicillin | Teicoplanin | Methicillin |
| MRSA1 | 31.25 | 250 | 250 | 250 | — |
| MRSA2 | 15.63 | 125 | 250 | 125 | — |
| MRSA3 | 15.63 | 125 | 250 | 125 | — |
| MRSA4 | 31.25 | 125 | 250 | 125 | — |
| MRSA5 | 31.25 | 125 | 250 | 125 | — |
| MRSA6 | 31.25 | 250 | 250 | 125 | — |
| MRSA7 | 15.63 | 250 | 250 | 250 | — |
| MRSA8 | 7.81 | 125 | 125 | 62.5 | — |
| MRSA8* | 15.63 | 125 | 125 | 31.25 | — |
| MRSA9 | 15.63 | 250 | 125 | 15.63 | — |
| MRSA10 | 15.63 | 250 | 250 | 31.25 | — |
| MRSA11 | 15.63 | 250 | 125 | 31.25 | — |
| MRSA11* | 15.63 | 31.25 | 250 | 62.5 | — |
| Average | 19.8346154 | 175.480769 | 211.538462 | 104.567692 | — |

### Example 6

### Bioassay of Macrolactin A

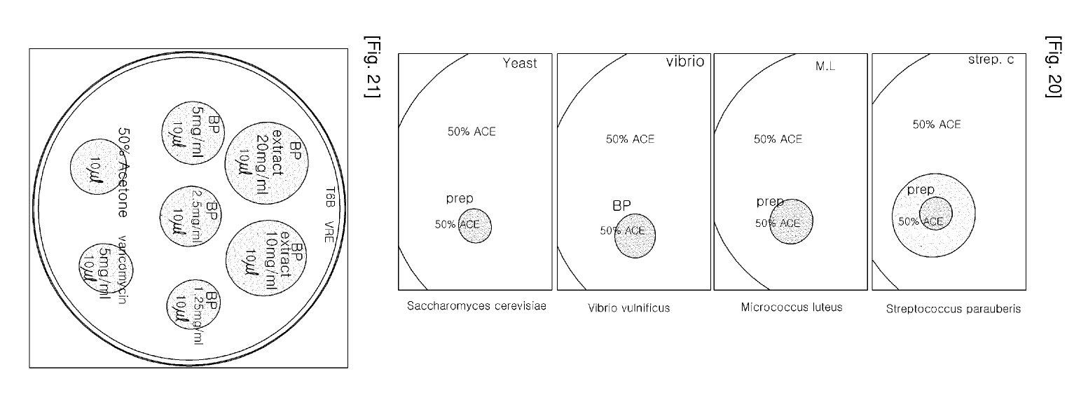

Bioassay was performed to measure the inhibitory effect of Macrolactin A produced by Bacillus polyfermenticus KJS-2, which is the strain of the present invention, on microorganisms. The strains used for the experiment were Saccharomyces cerevisiae, Vibrio vulnificus, Micrococcus luteus and Streptococcus parauberis. Each strain, except Vibrio vulnificus, was inoculated and cultured in TSB medium at 200 rpm for 16 hours at 37° C., and then a 0.5 ml (approximately 28*10⁹ cfu/ml) aliquot of the culture medium was spread over TSB agar medium. Vibrio vulnificus, was inoculated and cultured in TSB medium at 200 rpm for 16 hours at 25° C., which is its optimum growth conation, and then the amount of the culture medium was spread over the agar medium. The solvent required for the bioassay is a solvent, which can dissolve Macrolactin A, that has no toxicity against the strains used for the experiment. Methanol is a good solvent for Macrolactin A, but was not suitable for bioassay because it showed toxicity by itself against the strains used for experiment. 50% acetone, however, showed no toxicity against the three strains used for the experiment (refer to FIG. 20).

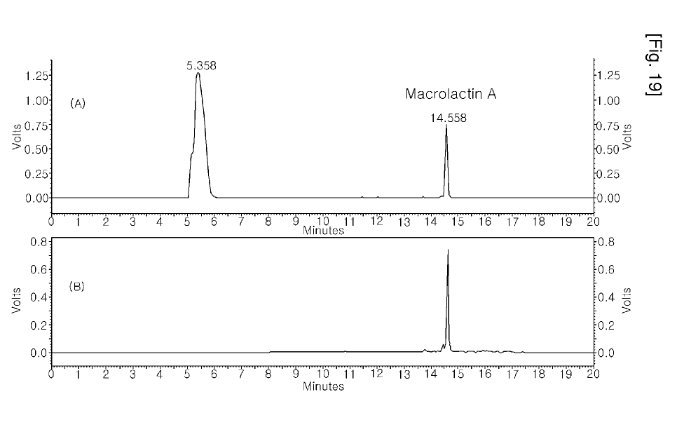

In FIG. 19, (A) is the HPLC chromatogram of 1 μl aliquot of 1 mg of Macrolactin A in 1 ml of 50% acetone that has been diluted 10 times, (B) is the HPLC chromatogram of 1 μl aliquot of 1 mg of Macrolactin A in 1 ml of methanol that has been diluted 10 times.

That is, the chromatogram is analyzed under the HPLC conditions of Step 2 of Example 2.

In summary of the experimental results, 50% acetone was used as a solvent for bioassay because 50% acetone showed no toxicity against the three strains used for experiment (FIG. 20), while the solubility of Macrolactin A in 50% acetone is similar to that in methanol (FIG. 19). 10 μl of the solution of Macrolactin A in 50% acetone (2.5 mg/ml) was tested and shown to have excellent antibiotic activity against the strains of Saccharomyces cerevisiae, Vibrio vulnificus, Micrococcus luteus and Streptococcus parauberis (refer to FIG. 20). In addition, 10 μl of Macrolactin A solutions (10 mg/ml, 2.5 mg/ml, 5 mg/ml, 10 mg/ml and 20 mg/ml in 50% acetone, respectively) were applied to VRE5, (the VRE5 used for the MIC experiment of the Example 5) which had been spread over TSB agar medium, and also 10 μl of 50% acetone and 100 of vancomycin solution (5 mg/ml in H₂O), respectively, were applied as control groups. The result showed that VRE5 used for the experiment was not inhibited by either the 50% acetone or the vancomycin solution (5 mg/ml), but inhibited by Macrolactin solutions with concentrations above 1.25 mg/ml (refer to FIG. 21).

### Example 7

### Inhibitory Effect on VRE in Liquid Medium

For the experiment with VRE, the strains were inoculated into a liquid medium upto a concentration of 1,000,000 cfu/mL and cultured either with (for strains to be tested) or without (for controls) Macrolactin A. The concentration of Macrolactin A was 50 μg/mL, and the following 11 strains used for the experiment were obtained from the Medical School of Donga University: VRE1, VRE2, VRE3, VRE4, VRE5, VRE6, VRE7, VRE8, VRE11, VRE914 and VRE915. As shown in the following Table, the control group (C) showed significant growth after 6 hours of culture as compared to 4 hours, while the group being tested showed significant growth retardation and inhibition; VRE8 and VRE11 showed notable growth retardation, but apparently with lower sensitivity than the other strains. Nine out of eleven strains tested showed remarkable growth inhibition. After 4 hours of culture, the average absorbance of the control group was 0.76, while that of the group cultured with Macrolactin A was 0.19, showing significant difference between the two groups. After 6 hours of culture, the control group showed rapid growth to the absorbance of 15, while the group cultured with Macrolactin A again showed significant growth inhibition to the absorbance of 0.26.

| | | | Macrolactin A | |
|---|---|---|---|---|
| Vancomycin- | Control group(C) OD | | Addition group OD | |
| resistant | Culture | Culture | Culture | Culture |
| enterococci | 4 hours | 6 hours | 4 hours | 6 hours |
| VRE1 | 0.987 | 1.678 | 0.216 | 0.207 |
| VRE2 | 0.924 | 1.621 | 0.279 | 0.391 |
| VRE3 | 0.656 | 1.283 | 0.240 | 0.314 |
| VRE4 | 0.415 | 1.300 | 0.166 | 0.180 |
| VRE5 | 0.594 | 1.533 | 0.176 | 0.153 |
| VRE6 | 0.690 | 1.531 | 0.171 | 0.179 |
| VRE7 | 0.809 | 1.649 | 0.173 | 0.133 |
| VRE8 | 0.841 | 1.559 | 0.228 | 0.589 |
| VRE11 | 0.913 | 1.528 | 0.236 | 0.507 |
| VRE14 | 0.790 | 1.355 | 0.078 | 0.050 |
| VRE15 | 0.694 | 1.497 | 0.145 | 0.131 |
| Average | 0.76 | 1.50 | 0.19 | 0.26 |

### Example 8

### Result of Measuring Specific Rotation

The specific rotation of Macrolactin A measured by “Gabriella”, etc was: [α]²²_{D} (c in MeOH)=−10.7 (0.68) (7-O-Malonyl Macrolactin A, a New Macrolactin Antibiotic from Bacillus subtilis Active against Methicillin-Resistant Staphylococcus aureus, Vancomycin-Resistant Enterococci, and a Small-Colony Variant of Burkholderia cepacia. Antimicrob. Agents Chemother. 50: 1701-1709, 2006). The specific rotation of Macrolactin A measured by “Yoo”, etc. was: [α]¹⁸_{D} (c in MeOH)=−20 (0.1) (Neuronal cell protection activity of macrolactin A produced by Actinomadura sp. J. Microbiol. Biotechnol. 7:429-434. 1997). The specific rotation of Macrolactin A measured by “William”, etc. was: [α]_{D} (c in MeOH)=−9.6 (1.86) (The macrolactins, a novel class of antiviral and cytotoxic macrolides from a deep-sea marine bacterium. J. Am. Chem. Soc. 111:7519-7524. 1989). The specific rotation of Macrolactin A measured by “Park”, etc. was: [α]²³_{D} (c in MeOH)=−10.36 (0.13). (Isolation and Characterization of Antimicrobial Substance Macrolactin A Produced from Bacillus amyloliquefaciens CHO104 Isolated from Soil. J. Microbiol. Biotechnol. 14:525-531, 2004).

The specific rotation of Macrolactin A of the present invention by Bacillus polyfermenticus KJS-2 was: [α]²²_{D} (c in MeOH)=−10 (4.0). This was different from the previous results, exemplifying the uniqueness as an optical isomer, while the specific rotation of saccharose, which was used as a control, showed a normal value of [α]²⁸_{D} (c in water)=64.038 (26). As a result, it can be concluded that the present invention has an isomer different from the Macrolactin A purified from the other strains.

1. A strain of Bacillus polyfermenticus KJS-2, which is a new strain producing Macrolactin A (Accession Number KCCM10769P).

2. A method of using the Macrolactin A of claim 1 as an antibiotic against vancomycin-resistant enterococci (VRE).

3. The method of claim 2, wherein MIC (Minimal inhibitory Concentration)_{>90} of the Macrolactin A is between 15.63 μg/ml and 31.25 μg/ml.

4. A method of using the Macrolactin A of claim 1 as an antibiotic against Methicillin-resistant Staphylococcus aureus (MRSA).

5. The method of claim 4, wherein MIC (Minimal Inhibitory Concentration)_{>90} of the Macrolactin A is between 7.81 μg/ml and 31.25 μg/ml.

6. A method of using the Macrolactin A of claim 1 as an antibiotic against infectious bacteria including Vibrio vulnificus and Streptococcus parauberis.
